(19) United States
(12) Patent Application Publication
(10) Pub. No.: US 2015/0050260 A1
(43) Pub. Date: Feb. 19, 2015

(54) PYRROLOQUINOLINE QUINONE BASED COMPOSITIONS AND USES

(71) Applicant: 4141 HOLDINGS, LLC, Weston, FL (US)

(72) Inventor: John H. Owoc, Weston, FL (US)

(21) Appl. No.: 14/461,301

(22) Filed: Aug. 15, 2014

Related U.S. Application Data
(60) Provisional application No. 61/884,355, filed on Sep. 30, 2013.
(60) Provisional application No. 61/866,835, filed on Aug. 16, 2013.

(51) Int. Cl. A61K 31/4745; A23L 1/30; A61K 31/197

(52) U.S. Cl. CPC A61K 31/4745; A61K 31/197; A23L 1/30; A23V 2002/00
USPC 424/94.1; 514/292; 514/690; 514/44 R

(57) ABSTRACT

The present invention provides pharmaceutical and dietary compositions in which pyrroloquinoline quinone (PQQ) and its derivatives are combined with other biologically active compounds to produce a synergistic beneficial effect beyond the effect produced by PQQ or its derivatives alone. The present invention also provides methods for providing various beneficial effects which comprise administering these compositions to a mammalian subject.

### CROSS-REFERENCE TO RELATED APPLICATIONS

This application claims priority to U.S. Provisional Application Ser. No. 61/884,355 filed on Sep. 30, 2013 and U.S. Provisional Application Ser. No. 61/866,835 filed on Aug. 16, 2013, and the contents of both are hereby incorporated in their entirety.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

Not Applicable

### BACKGROUND OF THE INVENTION

1. Field of the Invention

The present invention relates to pharmaceutical and dietary compositions comprising pyrroloquinoline quinone (PQQ), PQQ derivatives, and/or salts thereof. The present invention also relates to uses of the compositions for providing beneficial health effects.

2. Description of the Related Art

Bioactive compounds that stimulate mitochondrial biogenesis have many health benefits, including promoting longevity, improving energy utilization in mammals, and protecting cells from oxidative stress caused by reactive oxygen species. Pyrroloquinoline quinone (PQQ) is a vitamin-like compound reported to have numerous beneficial effects, including stimulating mitochondria biogenesis, protecting cells from oxidative stress, and improving reproductive ability. Previous studies have shown that mice and rats fed with diets lacking pyrroloquinoline quinone have reduced mitochondrial contents.

Creatine supplementation is widely used by athletes, body builders, wrestlers, sprinters, and others who wish to gain muscle mass to enhance sports fitness performance. Typical daily dosage of creatine supplementation is two to three times of the amount that could be obtained from a very-high-protein diet. Extensive researches have shown that oral creatine supplementation at a dose of 5 to 20 grams per day, which appears to be very safe and largely devoid of adverse side-effects, can effectively improve physiological response to resistance exercise and increase the maximal force production of muscles in both men and women. Creatine has been demonstrated to cause modest increases in strength in people with a variety of neuromuscular disorders (Tamopolsky M, Martin J (March 1999). “Creatine monohydrate increases strength in patients with neuromuscular disease”. Neurology 52 (4): 854-7).

Creatine supplementation can be used for the treatment of arthritis, congestive heart failure, disuse atrophy, gyrate atrophy, mitochondrial diseases, muscular dystrophy, and depression. A meta analysis found that creatine treatment increased muscle strength in muscular dystrophies, and potentially improved functional performance. Creatine has also been reported to decrease mutagenesis in DNA. (Rahimi, R. (2011). “Creatine Supplementation Decreases Oxidative DNA Damage and Lipid Peroxidation Induced by a Single Bout of Resistance Exercise,” Journal of Strength and Conditioning Research 25 (12): 3448-3455).

Amino acids are biologically important organic compounds comprised of amine (—NH2) and carboxylic acid (—COOH) groups, as well as side-chains. The key chemical elements of amino acids are carbon, hydrogen, oxygen, and nitrogen, while other chemical elements can also be found in the side-chains of certain amino acids. Many important proteinogenic and non-proteinogenic amino acids play a critical role in the body. For example, in the human brain, glutamate (standard glutamic acid) and gamma-amino-butyric acid (“GABA”, non-standard gamma-amino acid) are respectively the main excitatory and inhibitory neurotransmitters. Hydroxyproline, a major component of the connective tissue collagen, is synthesized from proline.

Amino acid glycine can be used to synthesize porphyrins used in red blood cells. Non-standard carnitine can be used in lipid transport. Nine of the twenty standard amino acids are essential for humans because these amino acids cannot be synthesized from other compounds by the human body; as a result, the essential amino acids must be supplied from the diet. Other amino acids may be conditionally essential for mammals (such as humans) of certain ages or medical conditions. Because of their biological significance, amino acids are important in nutrition. Tipton, Kevin, D. et al. examined the effect of the ingestion of amino acids after a bout of resistance exercise on net muscle protein synthesis. The results showed that oral ingestion of essential amino acids results in a change from net muscle protein degradation to net muscle protein synthesis after heavy resistance exercise in humans similar to that seen when the amino acids were infused (AJP—Endo Apr. 1, 1999 vol. 276 no. 4 E628-E634).

Elena Volpi et al. assessed whether nonessential amino acids are required in a nutritional supplement to stimulate muscle protein anabolism in the elderly. The study compared the effects of the ingestion of 18 g essential amino acids (EAA group: n=6, age 69+2 y; x+SD) or the ingestion of 40 g balanced amino acids (18 g essential amino acids+22 g nonessential amino acids, BAA group; n=8, age 71±2 y) on muscle protein metabolism in elderly people. Specifically, amino acids were given orally to healthy elderly volunteers in small boluses every 8-10 min for 3 hours. The results showed that essential amino acids are primarily responsible for the amino acid-induced stimulation of muscle protein anabolism in the elderly people. (Am J Clin Nutr August 2003 vol. 78 no. 2 250-258). Blomstrand, E. et al. studied the effect of branched-chain amino acids (BCAAs) on performance during sustained exercise. The results showed that both mental and physical performance was improved by an intake of BCCA during exercise (Eu J. App Physiol. (1991) 63: 83-88).

Shimomura et al. examined the effects of BCAAs supplementation on delayed-onset muscle soreness (DOMS) and muscle fatigue in humans induced by squat exercise. The results showed that BCAA supplementation prior to squat exercise decreased DOMS and muscle fatigue occurring for a few days after exercise. The results indicate that that BCAAs can be useful for muscle recovery following exercise (J. Nutr. February 2006 vol. 136 no. 2 529S-532S).

Melatonin, also known chemically as N-acetyl-5-methoxytryptamine, is a naturally occurring compound found in animals, plants, and microbes. In animals, circulating levels of the hormone melatonin vary in a daily cycle, thereby allowing the entrainment of the circadian rhythms of several biological functions. Melatonin is categorized by the US Food and Drug Administration (FDA) as a dietary supplement, not a drug. In humans, melatonin is produced by the pineal gland, a small endocrine gland located in the center of the brain but outside the blood-brain barrier. The melatonin signal forms part of the system that regulates the sleep-wake cycle by chemically causing drowsiness and lowering the body temperature; however, it is the central nervous system (specifically the suprachiasmatic nuclei, or SCN) that controls the daily cycle in most components of the paracrine and endocrine systems rather than the melatonin signal.

In addition to its function as synchronizer of the biological clock, melatonin was found to be a powerful free-radical scavenger and a wide-spectrum antioxidant. Studies have shown that melatonin plays a crucial part in the aging process and that it can act as an anti-aging agent when administered to older mice. It has been reported that administration of melatonin in elderly mice may reverse the change in expression of various genes thereby rejuvenating these elderly mice. Consuming melatonin can neutralize oxidative damage and delay the neurodegenerative process of aging. The administration of melatonin to mice has been shown to reduce the oxidative damage caused by aging, delay the inflammatory process, and promote longevity.

Melatonin receptors play a role in learning and memory, and melatonin can alter electrophysiological processes associated with memory, such as long-term potentiation (LTP). Melatonin can be used to treat or alleviate Alzheimer's disease, and has been reported to prevent neuronal death caused by exposure to the amyloid beta protein, a neurotoxic substance that accumulates in the brains of patients with Alzheimer's disease.

### BRIEF SUMMARY OF THE INVENTION

In one embodiment, the present invention provides a pharmaceutical or dietary 20 composition comprising pyrroloquinoline quinone (PQQ), and/or one or more derivatives of PQQ, and/or salts thereof; and one or more other biologically active compounds.

In one embodiment, the present invention provides a composition for oral administration, wherein the composition comprises pyrroloquinoline quinone (PQQ), PQQ derivative, and/or a salt thereof; and one or more biologically active compounds selected from creatine, creatine derivatives, creatylated peptides, coenzyme Q10, amino acids, melatonin, one or more compounds selected from lipoic acid, melatonin, ephedrine, caffeine, omega-3 fatty acids (fish oil), butein, piceatannol, fisetin, genistein, hydroxytyrosol, quercetin, isoliquiritigenin, casokinins, lactokinins, breakdown products of casein and whey, lactotripeptides (such as Val-Pro-Pro and Ile-Pro-Pro), nucleotides, oligonucleotides, monophosphates, diphosphates, triphosphates, and/or cyclic derivatives of nucleotides, short, medium, and/or long chain triglycerides; and any combination of ingredients selected from a) to g).

The present invention also provides uses of the compositions for providing beneficial health effects. In one embodiment, the method comprises administering to a subject an effective 10 amount of the compositions of the present invention.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### DETAILED DESCRIPTION

In one embodiment, the present invention provides a nutritional or dietary composition comprising pyrroloquinoline quinone (PQQ) and/or one or more derivatives of PQQ, and/or salts thereof; and one or more other biologically active compounds. Advantageously, the combination of PQQ and/or PQQ derivative(s) and/or salts thereof, with other biologically active compounds, produces a synergistic beneficial effect beyond the effect produced by PQQ or its derivatives alone. The invention includes a method of administering PQQ or its derivatives to a subject an effective amount of the compositions of the present invention.

### Pyrroloquinoline Quinone (PQQ) and PQQ Derivatives

(PQQ) has the following formula:

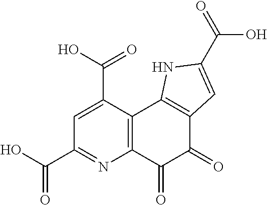 [SMILES: O=C(O)c1cc(C(=O)O)c2c(n1)C(=O)C(=O)c1cc(C(=O)O)[nH]c1-2]

PQQ derivatives include a compound having the following formula I:

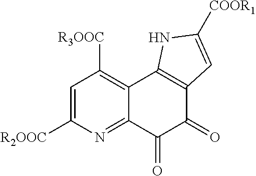

Wherein R1, R2, and R3 are, independently, a hydrogen atom, an alkyl group, an alkenyl group, a haloalkyl group, a benzyl group, or an alkoxycarbonylalkyl group, with the proviso that at least one of R1, R2, and R3 is not a hydrogen atom.

“Alkyl” means a linear saturated monovalent radical of one to sixteen carbon atoms or a branched saturated monovalent of three to sixteen carbon atoms. It may include hydrocarbon radicals of one to four or one to three carbon atoms, which may be linear. Examples include methyl, ethyl, propyl, 2-propyl, n-butyl, iso-butyl, tert-butyl, pentyl, and the like.

“Alkenyl” means a linear or branched C2-C16 hydrocarbon radical that comprises one or more carbon-carbon double bonds. Examples include propylenyl, buten-1-yl, isobutenyl, penten-1-yl, 2,2-methylbuten-1-yl, 3-methylbuten-1-yl, hexan-1-yl, hepten-1-yl, octen-1-yl, and the like. “Carboxyl” means the radical —C(0)OH.

“Haloalkyl” means alkyl substituted with one or more same or different halo atoms, e.g., —CH2C1, —CH2Br, —CF3, —CH2CH2C1, —CH2CC13, and the like.

“Amino” means the radical —NH2.

“Hydroxy” means the radical —OH.

“PQQ-based compounds” refers to PQQ, PQQ derivatives having the above formula and salts thereof.

In one embodiment, the present invention also includes salt forms of pyrroloquinoline quinone (PQQ) and PQQ derivatives. Salt forms of PQQ and PQQ derivatives useful according to the present invention include, but are not limited to, sodium, potassium, calcium, magnesium, zinc, and ethanolamine salts.

### Compositions

In one embodiment, the present invention provides a PQQ composition comprising pyrroloquinoline quinone (PQQ) and/or one or more derivatives of PQQ, and/or salts thereof and one or more other biologically active compounds. Advantageously, the combination of PQQ or PQQ derivative(s) and/or salts thereof, with other biologically active compounds, produces a synergistic beneficial effect beyond the effect produced by PQQ or its derivatives alone.

In certain embodiments, the present invention provides a composition comprising pyrroloquinoline quinone (PQQ) and/or a PQQ derivative, and/or salts thereof; and one or more biologically active compounds selected from creatine, amino acids, lipoic acid, melatonin, ephedrine, caffeine, omega-3 fatty acids (fish oil), butein, piceatannol, fisetin, genistein, hydroxytyrosol, quercetin, isoliquiritigenin, casokinins, lactokinins, breakdown products of casein and whey, and/or lactotripeptides (e.g., Val-Pro-Pro and Ile-Pro-Pro). Biologically active compounds may be natural or synthetic.

In certain embodiments, the present invention provides a composition comprising PQQ and/or its derivatives, and creatine and/or creatyl derivatives (such as creatyl derivatives described in U.S. Pat. No. 8,445,466, incorporated herein by reference), wherein the composition can be ingested by mammals or administered otherwise to elicit a beneficial synergistic effect beyond that one obtained with PQQ or its derivatives alone.

In certain embodiments, the present invention provides a composition comprising PQQ and/or its derivatives; creatine and/or creatyl derivatives (such as creatyl derivatives described in U.S. Pat. No. 8,445,466); and Coenzyme Q10 (CoQ10), wherein the composition can be ingested by mammals or administered otherwise to elicit a beneficial synergistic effect beyond that obtained with PQQ or its derivatives alone.

In one embodiment, the present invention provides a composition comprising PQQ, one or more PQQ derivatives, and/or salts thereof; and one or more biologically active compounds selected from melatonin and/or plant extracts containing melatonin, including but not limited to, feverfew (Tanacetum parthenium) and St John's wort (Hypericum perforatum); compounds that activate melatonin receptors; and antioxidant-containing food including, but not limited to, cherries, bananas, grapes, rice and cereals, herbs, olive oil, wine, and beer.

In one embodiment, the present invention provides a composition for oral administration, wherein the composition comprises PQQ, a PQQ derivative and/or a salt thereof, and one or more biologically active compounds selected from:

creatine, creatine derivatives, and/or creatylated peptides, coenzyme Q10, amino acids, melatonin, one or more compounds selected from lipoic acid, ephedrine, caffeine, omega-3 fatty acids (fish oil), butein, piceatannol, fisetin, genistein, hydroxytyrosol, quercetin, isoliquiritigenin, casokinins, lactokinins, breakdown products of casein and whey, lactotripeptides (such as Val-Pro-Pro and Ile-Pro-Pro), nucleotides, oligonucleotides, monophosphates, diphosphates, triphosphates and/or cyclic derivatives of nucleotides, short, medium, and/or long chain triglycerides, and any combinations thereof.

In certain embodiments, the compositions of the present invention can be formulated into nutritional supplements and pharmaceutical compositions including, but not limited to, tablet or capsule dosage forms, aqueous and emulsion injectable formulations, aqueous intra-nasal, and/or intra-ocular gel systems.

In one embodiment, the present invention provides a synergistic composition comprising PQQ, one or more PQQ derivatives, and/or a salt thereof; and creatine and/or one or more creatine derivatives. In one embodiment, the composition comprises creatyl peptides describe in U.S. Pat. No. 8,445,466, which is herein incorporated by reference in its entirety.

In one embodiment, the present invention provides synergistic composition comprising PQQ, one or more PQQ derivatives, and/or salts thereof; and one or more proteinogenic and non-proteinogenic amino acids.

In certain embodiments, the present invention provides nutritional supplements in tablet or capsule dosage forms, aqueous and emulsion injectable formulations, aqueous intra-nasal or intra-ocular gel systems that can be used to treat Huntington's disease, treat pediatric congestive heart failure (CHF), reduce oxidation of low-density lipoproteins, reduce migraine headaches, treat cancer and provide relief from cancer treatment side-effects, lower systolic blood pressure and diastolic blood pressure, reduce oxidation and DNA double strand breaks thus extending lifespan, improve mitochondrial respiratory control or stimulate mitochondrial biogenesis, increase longevity, improve energy utilization, provide protection from reactive oxygen species (ROS), and treat diseases associated with mitochondria dysfunction.

In one embodiment, the present invention provides a synergistic composition comprising PQQ, and/or one or more PQQ derivatives, and/or salts thereof; and one or more biologically active compounds selected from anti-aging compounds that activate SIRT genes; ACE genes; transcription factors such as DAF 16; ephedrine; caffeine; omega-3 fatty acids (fish oil); butein; piceatannol; fisetin; quercetin; isoliquiritigenin; casokinins; lactokinins; breakdown products of casein and/or whey; and lactotripeptides (such as Val-Pro-Pro and Ile-Pro-Pro).

In certain embodiments, the compositions of the present invention may be used to stimulate the expression of selective internal radiation therapy (SIRT) genes thereby improving insulin sensitivity; to induce the expression of the angiotensin converting enzyme (ACE) genes thereby increasing blood pressure; to induce the expression of DAF-16 genes, which are a group of genes involved in biological processes involved in aging, immunity, and responses to physical or environmental stress. Expression of ACE genes results in vasoconstriction, which can result in higher maximum oxygen uptake (VO2 max) associated with endurance in long distance sports events.

In one embodiment, the present invention excludes compositions disclosed in US Patent Application Publication No. 2009/0192312 and US Patent Application Publication No. 2007/0293572.

Composition 1

In one embodiment, the present invention provides an oral liquid composition (Ready-to-Drink or RTD) comprising PQQ and/or a PQQ derivative and/or a salt thereof; wherein the composition has a pH of about 3.5 to 6.5 and is substantially stable at room temperature for normal warehouse storage conditions.

In one embodiment, the composition comprises a suitable aqueous solvent or vehicle, a non-aqueous vehicle, a preservative, a physical stabilizing ingredient, one or more surfactants, and/or one or more buffer salts that can render the composition pH stable.

The composition may also contain one or more biologically active compounds including, but not limited to, creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and lactotripeptides (such as Val-Pro-Pro and Ile-Pro-Pro) which may be produced by probiotic Lactobacillus (such as Lactobacillus helveticus) or derived from casein.

The composition may also contain nucleosides, oligonucleotides, the monophosphates, diphosphates, triphosphates and/or cyclic derivatives of nucleosides (such as ATP, UTP, etc.), and amino acids, vitamins and vitamin-like isoprenoids, and/or peptides.

In one embodiment, the composition further comprises lipids, short, medium and/or long chain triglycerides, starches, carbohydrates, polyols, minerals, electrolytes, amino trace elements, colorings, flavors, artificial sweeteners, and/or anti-oxidants.

Composition 2

In one embodiment, the present invention provides an oral liquid composition for buccal sublingual administration comprising PQQ, and/or a PQQ derivative, and/or a salt thereof; wherein the composition has a pH of about 3.5 to 6.5 and is substantially stable at room temperature for normal warehouse storage conditions.

The composition may also contain one or more biologically active compounds including, but not limited to, creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and lactotripeptides (such as Val-Pro-Pro and Ile-Pro-Pro), which may be produced by probiotic Lactobacillus (such as Lactobacillus helveticus) or derived from casein.

The composition may also comprise a suitable aqueous solvent or vehicle, a non-aqueous vehicle, a preservative, a physical stabilizing ingredient, one or more surfactants, and/or one or more buffer salts that can render the composition pH stable.

The composition may also contain nucleosides, oligonucleotides, the monophosphates, diphosphates, triphosphates and/or cyclic derivatives of nucleosides (such as ATP, UTP, etc.), amino acids, vitamins and vitamin-like isoprenoids, and/or peptides.

In one embodiment, the composition further comprises lipids, short, medium and/or long chain triglycerides, starches, carbohydrates, polyols, minerals, electrolytes, amino trace elements, colorings, flavors, artificial sweeteners, and/or anti-oxidants.

Composition 3

In one embodiment, the present invention provides an oral solid composition in the form of a capsule (such as LiCap®) with a liquid composition as fill material containing from about 1% (w/w) to about 20% (w/w) of water; wherein the liquid fill material has a pH of about 3.5 to 6.5 and is substantially stable at room temperature for normal warehouse storage conditions.

In certain embodiments, the composition comprises a suitable lipophilic solvent or vehicle, a hydrophilic non-aqueous vehicle, about 1% (w/w) to about 20% (w/w) of water, a preservative, a physical stabilizing ingredient, one or more surfactants, and/or one or more buffer salts that can render the composition pH stable.

The composition may also contain creatine, creatine derivatives and/or creatylated peptides, amino acids, melatonin, ephedrine, caffeine, omega-3 fatty acids (fish oil), butein, 11-piceatannol, fisetin, quercetin, isoliquiritigenin, casokinins, lactokinins, breakdown products of casein and whey, and/or lactotripeptides (such as Val-Pro-Pro and Ile-Pro-Pro) which may be produced by probiotic Lactobacillus (such as Lactobacillus helveticus) or derived from casein.

The composition may also contain nucleosides, oligonucleotides, the monophosphates, diphosphates, triphosphates and/or cyclic derivatives of these nucleosides (such as ATP, UTP, etc.), amino acids, vitamins and vitamin-like isoprenoids, and/or peptides.

In one embodiment, the composition further comprises lipids, medium and/or short chain triglycerides, starches, polyols, carbohydrates, minerals, electrolytes, amino trace elements, colorings, and/or anti-oxidants.

Composition 4

In one embodiment, the present invention provides an oral liquid composition comprising 1 gram to 100 grams of protein and 1 gram to 100 grams of carbohydrates per serving, wherein the composition comprises PQQ and/or a PQQ derivative, and/or a salt thereof; wherein the liquid composition is substantially stable at room temperature for normal warehouse storage conditions.

The composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro) which may be produced by probiotic Lactobacillus (Lactobacillus helveticus) or derived from casein.

In certain embodiments, the composition comprises an acid stable protein isolate or a combination or blend of protein isolates, concentrates and hydrolyzates and caseins in micellar forms, a suitable aqueous solvent or vehicle, a non-aqueous vehicle, a preservative, a physical stabilizing ingredient, one or more surfactants, and/or one or more buffer salts that can render the composition pH stable.

The composition may also contain nucleosides, oligonucleotides, the monophosphates, diphosphates, triphosphates and/or cyclic derivatives of nucleosides (such as ATP, UTP, etc.), amino acids, and/or vitamins and vitamin-like isoprenoids, peptides.

In one embodiment, the composition further comprises lipids, short, medium and/or long chain triglyePridPs, stgrohes, oarhnhydrgtes, pnlynlc, mineralc, electrolytec, amino trace elementc, colorings, flavors, artificial sweeteners, and/or anti-oxidants.

Composition 5

In one embodiment, the present invention provides an aqueous injectable composition for administration to animals including humans, wherein the composition is isotonic and sterile, and comprises PQQ and/or a PQQ derivative and/or a salt thereof; wherein the injectable preparation has a pH of about 3.5 to 6.5, and is substantially stable at room temperature for normal warehouse storage conditions.

The composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro).

In one embodiment, the composition further comprises a suitable aqueous solvent, a preservative, a physical stabilizing ingredient, and/or one or more buffer salts that can render the composition pH stable.

The composition may also contain nucleosides, oligonucleotides, monophosphates, diphosphates, triphosphates and/or cyclic derivatives of nucleosides (such as ATP, UTP, etc.), amino acids, peptides, proteins, and/or carbohydrates.

Composition 6

In one embodiment, the present invention provides an emulsion injectable composition for administration to animals including humans, wherein the composition is isotonic and sterile and comprises PQQ, and/or a PQQ derivative, and/or a salt thereof; wherein the injectable preparation has a pH of about 3.5 to 6.5, and is substantially stable at room temperature for normal warehouse storage conditions.

The composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro) produced by probiotic Lactobacillus (such as Lactobacillus helveticus) or derived from casein.

The composition may also comprise a suitable aqueous solvent, pharmaceutically acceptable oils (sesame, olive, castor, peanut, cotton seed, short, medium and long chain triglycerides, etc.), a natural emulsifier (such as lecithin or any other synthetic emulsifier such as polysorbate or ethoxylated glyceride type), a preservative, a physical stabilizing ingredient, and/or one or more buffer salts that can render the composition pH stable.

The composition may also contain nucleosides, oligonucleotides, monophosphates, diphosphates, triphosphates and/or cyclic derivatives of nucleosides (such as ATP, UTP, etc.), amino acids, peptides, proteins, and/or carbohydrates.

Composition 7

In one embodiment, the present invention provides a gel topical composition for skin application in animals including humans, wherein the composition is clear or slightly opaque and has a gel consistency so that it can be spread on skin surface, and wherein the composition comprises PQQ, and/or a PQQ derivative, and/or a salt thereof, wherein the gel has a pH of about 3.5 to 6.5, and is substantially stable at room temperature for normal warehouse storage conditions.

The composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, lactotripeptides (such as Val-Pro-Pro and Ile-Pro-Pro).

The composition can further comprises a suitable aqueous solvent, a preservative, a polymer for imparting consistency, a physical stabilizing ingredient, and/or one or more buffer salts that can render the composition pH stable. The composition may also contain nucleosides, oligonucleotides, monophosphates, diphosphates, triphosphates, and/or cyclic derivatives of nucleosides (such as ATP, UTP, etc.), amino acids, vitamins and vitamin-like isoprenoids, peptides, proteins, and/or carbohydrates.

Composition 8

In one embodiment, the present invention provides a cream topical composition for skin application in animals including humans, wherein the composition is of an emulsion formulation or an opacified gel formulation and has a creamy consistency so that it can be spread on skin surface, wherein the composition comprises PQQ and/or a PQQ derivative and/or a salt thereof, and wherein said composition has a pH of about 3.5 to 6.5, and is substantially stable at room temperature for normal warehouse storage conditions.

In certain embodiments, the composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and IlePro-Pro).

In certain embodiments, the composition can further comprise a suitable aqueous solvent, a preservative, a physical stabilizing ingredient, a surfactant, moisturizers, and/or one or more buffer salts that can render the composition pH stable.

The composition may also contain nucleosides, oligonucleotides, the monophosphates, diphosphates, triphosphates and/or cyclic derivatives of nucleosides (such as ATP, UTP, etc.), amino acids, vitamins and vitamin-like isoprenoids, and/or peptides.

In one embodiment, the composition may further comprise lipids, short, medium and long chain triglycerides, starches, carbohydrates, polyols, minerals, electrolytes, amino trace elements, colorings, flavors, artificial sweeteners, and/or anti-oxidants.

Composition 9

In one embodiment, the present invention provides a deep-penetrating transdermal composition for application in animals including humans, wherein the composition is a solution, a gel-like, an emulsion-like, or an opacified gel-like formulation, wherein the composition has a consistency so that it can be spread on skin surface, wherein the composition comprises PQQ, and/or a PQQ derivative, and/or a salt thereof, wherein the transdermal composition is substantially stable at room temperature for normal warehouse storage conditions.

In certain embodiments, the composition may also contain creatine, creatine derivatives and/or creatylated peptides, amino acids, melatnnin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro).

In certain embodiments, the composition can further comprise a suitable aqueous solvent, a non-aqueous solvent, one or more penetrating enhancers, a preservative, a physical stabilizing ingredient, one or more surfactants, moisturizers, and/or one or more buffer salts that can render the composition pH stable.

In certain embodiments, the composition may also contain nucleosides, oligonucleotides, monophosphates, diphosphates, triphosphates and/or cyclic derivatives of these nucleosides (such as ATP, UTP, etc.), amino acids, vitamins and vitamin-like isoprenoids, peptides, proteins, lipids, short, medium and/or long chain triglycerides, and/or and carbohydrates.

Composition 10

In one embodiment, the present invention provides a transdermal patch delivery system comprising a liner, an adhesive, a backing and an aqueous liquid reservoir composition. In one embodiment, the aqueous liquid reservoir composition is a solution or a suspension comprising PQQ, and/or a PQQ derivative, and/or a salt thereof; wherein the transdermal patch is substantially stable at room temperature for normal warehouse storage conditions.

In certain embodiments, the composition may also contain creatine, creatine derivatives and/or creatylated peptides, amino acids, melatonin, ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro).

In certain embodiments, the composition further comprises a suitable aqueous solvent, a non-aqueous solvent, one or more penetrating enhancers, a preservative, a physical stabilizing ingredient, one or more surfactants, and/or one or more buffer salts that can render the composition pH stable.

The composition may also contain nucleotides, oligonucleotides, monophosphates, diphosphates, triphosphates 2nd/nr cyclic derivatives of these miclentidec, amino acids, vitamins and vitamin-like isoprenoids, peptides, proteins, and/or carbohydrates.

Composition 11

In one embodiment, the present invention provides a composition for oral administration of a biologically active form of pyrroloquinoline quinone or its derivatives to a mammalian subject, wherein the composition further comprises one or more biologically active compounds selected from:

a) creatine, creatine derivatives and/or creatylated peptides;

b) amino acids;

c) melatonin; and

d) one or more compounds selected from ephedrine, caffeine, omega-3 fatty acids (fish oil), butein, piceatannol, fisetin, quercetin, isoliquiritigenin, casokinins, lactokinins, breakdown products of casein and whey, and lactotripeptides (such as Val-Pro-Pro and Ile-Pro-Pro).

In one embodiment of the composition, PQQ, PQQ derivatives, and/or salts thereof, are present in the composition in any amount between about 0.01% and about 10% by weight based on the total weight of said composition, including all percentages and ranges of percentages therebetween.

In one embodiment, the composition may further comprise one or more adjuvant materials, including but not limited to, flavoring agents, colorants, viscosity modifiers, preservatives, chelating agents, antioxidants, surface modifiers, and other nutritional adjuvant materials. Other nutritional adjuvant materials include any substance that is generally recognized as promoting the health or function of a mammalian organism, including humans, or benefiting a composition useful thereof in terms of its efficacy, appearance, stability, consistency, aroma, or viscosity. Such substances include, but are not limited to, other non-essential amino acids and their salts, vitamins, minerals, essential fatty acids, enzymes, mono-glycerides, di-glycerides, tri-glyceride ester oils (including, for example vegetable oils and animal fats) emulsifiers, hydrolyzed proteins, whey protein, stabilizers, flow modifiers, viscosity improvers, chelating agents, enzymes, and surfactants, whether anionic, cationic or nonionic.

In one embodiment, the total amount of the one or more nutritional adjuvant materials above present in a composition may be any amount between about 0.01% and about 50% by weight based on the total weight of said composition, including all percentages and ranges of percentages therebetween.

In one embodiment, the composition can also comprise one or more natural beverages. A natural beverage, as used herein, is a beverage suitable for human or animal consumption which contains the pulp, juice or any other constituent of a naturally-occurring fruit, vegetable, or animal product whether from the wild, cultured, cultivated on a farm or otherwise domesticated by humans.

Natural beverages include without limitation materials such as milk products, soy products, ice cream, yoghurt, citrus fruit juices, non-citrus fruit juices, and vegetable juices, or components of any of the foregoing, wherein said natural beverages are present in any effective amount to impart flavor to the compositions, which may be any amount between about 0.1% and about 99% by weight based on the total weight of said composition, including all percentages and ranges of percentages there between. In addition to ingredients containing other adjuvant materials, a composition according to this disclosure may alternatively comprise one or more synthetic beverages. A synthetic beverage is any beverage which is not a natural beverage. A composition according to this disclosure may be made quite palatable to a mammalian subject, including human subjects, when it is administered orally in an aqueous mixture. Typical 20 serving sizes may be any serving size in the range of about 1 milligram to about 50 grams, in an aqueous solution that is from about 20 ml to about 2500 ml in volume. In one embodiment, an aqueous composition of the present invention is limited only by the solubility limit of the PQQ and its derivatives, which may exceed 50 grams per liter.

In certain embodiments, concentrations at or near the solubility limit are herein provided by contacting excess amounts of the PQQ and its derivatives in contact with water or an aqueous solution to provide a solution saturated with PQQ and its derivatives. Such saturated solutions may then be diluted slightly, to afford a concentrate from which other PQQ and its derivatives containing compositions may be conveniently provided.

In one embodiment of the composition, creatine and/or creatine derivatives are present in any amount between 0.001% and 20% by weight based on the total weight of said composition.

In one embodiment of the composition, amino acids are present in any amount between 0.001% and 20% by weight based on the total weight of said composition.

In one embodiment of the composition, melatonin is present in any amount between 0.001% and 10% by weight based on the total weight of said composition.

In one embodiment of the composition, the total weight of ephedrine, caffeine, omega-3 fatty acids (fish oil), butein, piceatannol, fisetin, quercetin, isoliquiritigenin, casokinins, lactokinins, breakdown products of casein and whey, and/or lactotripeptides (such as Val-Pro-Pro and Ile-Pro-Pro) produced by probiotic Lactobacillus (such as Lactobacillus helveticus) or derived from casein are present in any amount between 0.001% and 20% by weight based on the total weight of the composition.

In one embodiment, the composition further comprises an anti-microbial preservative present in an effective amount to inhibit microbial growth. Preservatives useful according to the present invention include, but are not limited to, esters of para-hydroxy benzoic acid, propionates, and one or more sorbate salts.

### Therapeutic Methods

Also provided are therapeutic uses of the compositions of the present invention. In one embodiment, the method comprises administering to a subject an effective amount of the compositions of the present invention.

In one embodiment, the present invention provides a method for providing prophylactic supplementation for patients in high stroke-risk categories comprising the step of orally, intra-nasally or intra-ocularly administering an effective amount of a composition of the present invention to a mammalian subject.

In certain embodiments, the compositions of the present invention can be used to treat Huntington's disease, treat pediatric congestive heart failure (CHF), reduce oxidation of low-density lipoproteins, treat migraine headaches, treat cancer and relieve from cancer treatment side-effects, lower systolic blood pressure and diastolic blood pressure, reduce oxidation and DNA double strand breaks thus extending lifespan, improve mitochondrial respiratory control, stimulate mitochondrial biogenesis, increase longevity, improve energy utilization, protect from reactive oxygen species (ROS), and treat diseases associated with mitochondria dysfunction.

In one embodiment, the present invention provides a method for treating cortical damage to a quilieet (q11eh qq. children and adolescents) with traumatic' hrnin injury comprising the step of orally, intra-nasally or intra-ocularly administering an effective amount of a composition of the present invention to a mammalian subject.

In one embodiment, the present invention provides a method for activating the expression of SIRT genes, ACE genes and/or DAF 16 genes, comprising the step of orally, intra-nasally or intra-ocularly administering an effective a composition of the present invention to a mammalian subject.

In one embodiment, the present invention provides a method of protecting nuclear and mitochondrial DNA comprising the step of orally, intra-nasally or intra-ocularly administering an effective amount of a composition of the present invention to a mammalian subject.

In one embodiment, the present invention involves chorionic administration of a composition of the present invention. In one embodiment, the present invention involves administering a composition of the present invention for any period of longer than 10 days, including but not limited to, longer than 15 days, longer than 20 days, longer than 25 days, longer than 30 days, longer than 2 months, longer than 3 months, longer than 6 months, and longer than 1 year.

In another embodiment, the present invention involves acute administration of a composition of the present invention. In one embodiment, the present invention involves administering a composition of the present invention for any period of shorter than 30 days, including but not limited to, shorter than 30 days, shorter than 25 days, shorter than 20 days, shorter than 15 days, shorter than 10 days, shorter than 5 days, shorter than 4 days, shorter than 3 days, or shorter than 2 days.

In certain embodiments, the composition of the present invention is administered orally, intra-nasally, or intra-ocularly.

The term “subject,” as used herein, describes an organism, including mammals such as primates, to which treatment with the compositions according to the present invention can be provided. Mammalian species that can benefit from the disclosed methods of treatment include, but are not limited to apes, chimpanzees, orangutans, humans, monkeys; and domesticated animals such as dogs, cats, horses, cattle, pigs, sheep, goats, chickens, mice, rats, guinea pigs, and hamsters.

The term “effective amount,” as used herein, refers to an amount that is capable of preventing ameliorating or treating a disease, disorder or condition.

The term “treatment” or any grammatical variation thereof (e.g., treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

Formulations and Administration

In one embodiment, the composition of the present invention is buffered at a pH of about 1.5 to about 6.5, or any pH therebetween.

The compositions of the present invention can be formulated into nutritional supplements, aqueous and emulsion injectable formulations, aqueous clear gel systems, creams and lotions, active-in-adhesive transdermal systems, and aqueous liquid-reservoir transdermal patches. Specifically exemplified herein are compositions for oral use. The subject invention further provides compositions for injection as well as for topical administration.

In a specific embodiment, the subject invention provides aqueous compositions suitable for oral administration to mammals including, without limitation, humans.

A composition as provided herein may be administered chronically. As used herein, “chronically” means repeated ingestion over a period of several days, several weeks, even several months, or longer. Acute (non-chronic) administration may also be utilized.

In one embodiment, the subject invention provides aqueous compositions having a pH in the range of about 1.5 to about 6.5. The pH can be obtained by using appropriate amounts of strong or weak acids or bases including, without limitation, aqueous mineral acids including HCl, H₃PO₄, and bases including sodium hydroxide, ethanolamines, etc. Preferably, the pH is from about 3.0 to about 6.5.

In certain embodiments, the total concentration of PQQ, PQQ derivative(s) and/or salts thereof in an aqueous solution provided hereby may be any amount between about 0.1% and about 90% by weight based on the total weight of the aqueous solution, including all percentages and ranges of percentages therebetween.

A composition according to this invention may also include other ingredients such as, for example, flavoring agents, colorants, viscosity modifiers, preservatives, chelating agents, antioxidants, surface modifiers and other nutritional adjuvant materials. Other materials include any substance that is generally recognized as promoting the health or function of a mammalian organism, including humans, or benefiting a composition useful thereof in terms of its efficacy, appearance, stability, consistency, aroma, or viscosity. Such substances include, for example, other amino acids and their salts, vitamins, minerals, fatty acids, enzymes, mono-glycerides, di-glycerides, tri-glyceride ester oils (including, for example, vegetable oils and animal fats) emulsifiers, hydrolyzed proteins, whey protein, stabilizers, flow modifiers, viscosity improvers, chelating agents, enzymes, and surfactants (whether anionic, cationic or nonionic). The total amount of these materials in a composition can be any amount between about 0.01% and about 50% by weight based on the total weight of said composition, including all percentages and ranges of percentages therebetween.

A composition according to this invention may also comprise one or more natural or synthetic beverages. For example, a natural beverage may contain the pulp, juice or any other constituents of a naturally-occurring fruit, vegetable, or animal product whether from the wild, cultured, cultivated on a farm or otherwise domesticated.

Natural beverages include, without limitation, materials such as milk products, soy products, ice cream, yogurt, citrus fruit juices, non-citrus fruit juices, and vegetable juices, or components of any of the foregoing, wherein said natural beverages are present in any effective amount to impart flavor to the compositions, which may be any amount between about 0.1% and about 99% by weight based on the total weight of said composition, including all percentages and ranges of percentages there between.

The compositions of the subject invention can be formulated for a variety of modes of administration. These formulations include, but are not limited to, compositions for oral administration, aqueous injectable formulations, injectable emulsion compositions, gel formulations, cream formulations, transdermal systems, transdermal patch systems, liquid buccal sublingual solutions, oral solid compositions, and oral liquid composition with protein.

### EXAMPLES

Following are examples that illustrate embodiments and procedures for practicing the invention. These examples should not be construed as limiting. All percentages are by weight and all solvent mixture proportions are by volume unless otherwise noted.

### Example 1

### Ready to Drink

| INGREDIENTS (Ready-to-Drink Formulation) | % w/w |
|---|---|
| Purified water | 97.1 |
| PQQ and/or derivative | 1.00 |
| Gamma Butyrobetaine | 0.0156 |
| Glycerin | 1.067 |
| Anserine | 0.052 |
| Caffeine | 0.06 |
| Magnesium Tanshinoate | 0.0000009 |
| L-Leucine | 0.104 |
| L-Isoleucine | 0.052 |
| L-Valine | 0.0208 |
| l,3-di-n-propyl-7-propargylxanthine | 1 × 10⁻¹° |
| Citric acid to pH 3.5 | 0.179 |
| Sodium benzoate | 0.052 |
| Potassium sorbate | 0.01 |
| Bis picolinate vanadium | 0.0000002 |
| Salt | 0.005 |
| Potassium phosphate dibasic | 0.0206 |
| Sodium Erythorbate | 0.000001 |
| Nisaplin | 0.000001 |
| Sucralose | 0.073 |
| Malic acid | 0.083 |
| Flavor Melon | 0.105 |

### Example 2

### Liquid Buccal Sublingual Solution

| INGREDIENTS | % w/w |
|---|---|
| PQQ and/or derivative | 2.10 |
| Peptides | 3.00 |
| AMP | 12.50 |
| UTP | 0.10 |
| Alcohol USP | 45.0 |
| Buffer Salt(s) | QS to adjust pH |
| Purified Water | QS to 100 |

### Example 3

### Liquid Buccal Sublingual Solution

| INGREDIENTS | % w/w |
|---|---|
| PQQ and/or derivative | 2.10 |
| Peptides | 3.00 |
| AMP | 12.50 |
| UTP | 0.10 |
| Alcohol USP | 50.0 |
| Benzyl alcohol | 1.00 |
| Buffer Salt(s) | QS to adjust pH |
| Purified Water | QS to 100 |

### Example 4

### Liquid Buccal Sublingual Solution

| INGREDIENTS | % w/w |
|---|---|
| PQQ and/or derivative | 2.10 |
| Peptides | 3.00 |
| AMP | 12.50 |
| UTP | 0.10 |
| Propylene Glycol | 20.0 |
| Alcohol USP | 40.0 |
| Polysorbate 80 | 5.0 |
| Benzyl alcohol | 1.00 |
| Buffer Salt(s) | QS to adjust pH |
| Purified Water | QS to 100 |

### Example 5

### Fill Material Composition for Capsule

| INGREDIENTS | % w/w |
|---|---|
| PQQ and/or derivative | 2.10 |
| Medium chain triglyceride | 15.0 |
| Peptides | 3.00 |
| AMP | 12.50 |
| UTP | 0.10 |
| Oleic Acid | 52.0 |

### Example 6

### Fill Material Composition for Capsule

| INGREDIENTS | % w/w |
|---|---|
| PQQ and/or derivative | 2.10 |
| Peptides | 3.00 |
| AMP | 12.50 |
| UTP | 0.10 |
| Polysorbate 80 | 25.0 |
| PEG-40 Hydrogenated Castor Oil | 38.00 |
| PEG esters and monoglycerides | 15.0 |

### Example 7

### Fill Material Composition for Capsule

| INGREDIENTS | % w/w |
|---|---|
| PQQ and/or derivative | 2.10 |
| Peptides | 3.00 |
| AMP | 12.50 |
| UTP | 0.10 |
| PEG-400 | 45.0 |
| PEG esters and monoglycerides | 9.00 |
| Polysorbate 80 | 20.0 |

### Example 8

### Medium Range pH RTD Protein Blend Formulations

| INGREDIENTS | % w/w | Per 16 oz Serving |
|---|---|---|
| PQQ and/or derivative | 1.00 | 2.10 |
| Whey Protein Isolate | 6.000 | 30.00 |
| Whey Protein Concentrate | 0.640 | 3.20 |
| Whey Hydrolysate | 0.320 | 1.60 |
| Micellar casein | 0.320 | 1.60 |
| Casein Protein Hydrolysate | 0.000 | 0.00 |
| Potassium Chloride | 0.076 | 0.38 |
| Ascorbic Acid | 0.012 | 0.06 |
| Vitamin E TPGS | 0.052 | 0.26 |
| Riboflavin 100 | 0.000 | 0.00000010 |
| Niacin | 0.000 | 0.0020 |
| Pyrodoxine HC1 | 0.000 | 0.000007 |
| Calcium Panthothenate | 0.000 | 0.0011 |
| Magnesium Maleate | 0.020 | 0.1000 |
| d-ribose | 0.040 | 0.2000 |
| Lecithin | 0.600 | 3.00 |
| Saflower Oil | 1.200 | 6.00 |
| Sunflower Oil | 1.200 | 6.00 |
| Medium Chain Triglycerides | 0.800 | 4.00 |
| L-Glutamine | 0.025 | 0.13 |
| Glucose Polymers ( Rice trin) | 0.800 | 4.00 |
| Waxy Maize Starch | 1.000 | 5.00 |
| High Amylose Starch (Amylose ADP11P) | 0.100 | 0.50 |
| Magnesium Citrate | 0.124 | 0.62 |
| Microcrystalline Cellulose | 0.100 | 0.50 |
| Malic Acid | 0.140 | 0.70 |
| Citric acid to pH 6.5 | 0.566 | 2.83 |
| Sodium Citrate to pH 6.5 | 0.140 | 0.70 |
| Sucralose | 0.011 | 0.06 |
| Glycerin | 3.000 | 15.00 |
| Sodium Benzoate | 0.090 | 0.45 |
| Potassium Sorbate | 0.190 | 0.95 |
| Water | QS | QS |

### Example 9

### Low pH RTD Protein Formulations

| INGREDIENTS | Per 16 oz serving | % w/w |
|---|---|---|
| PQQ and/or derivative | 2.10 | 0.25 |
| Whey Protein Isolate Acid Stable | 44.44 | 9.26 |
| Sucralose | 0.12 | 0.025 |
| Sodium EDTA | 0.24 | 0.050 |
| Potassium Sorbate | 0.96 | 0.200 |
| Sodium Benzoate | 0.48 | 0.100 |
| Citric Acid to pH 3.5 | QS | QS |
| Malic Acid to pH 3.5 | QS | QS |
| Water | 433.8 | 90.37 |
| Total | 480 | 100 |

### Example 10

### Low pH RTD Protein Formulations

| INGREDIENTS | Per 16 oz serving | % w/w |
|---|---|---|
| PQQ-and/or derivative | 2.10 | 0.25 |
| Whey Protein Isolate Acid Stable | 44.44 | 9.26 |
| Sucralose | 0.12 | 0.025 |
| Waxy Maize Starch | 4.80 | 1.00 |
| Glucose Polymers ( Rice trin) | 0.96 | 0.20 |
| Na EDTA | 0.24 | 0.050 |
| Potassium Sorbate | 0.96 | 0.200 |
| Sodium Benzoate | 0.48 | 0.100 |
| Citric Acid to pH 3.5 | QS | QS |
| Malic Acid to pH 3.5 | QS | QS |
| Water | QS | QS |
| TOTAL | 480 | 100 |

### Example 11

### Aqueous Injectable Formulations

| INGREDIENTS | % w/w |
|---|---|
| PQQ and/or derivative | 1.00 |
| AMP | 12.5 |
| UTP | 0.10 |
| Amino Acids | 3.0-7.0 |
| Polysorbate 80 | 0.40 |
| Sodium CMC | 0.50 |
| Sodium Chloride | 0.90 |
| Benzyl alcohol | 0.90 |
| Buffer Salt(s) | QS to adjust to pH 3.5-6.5 |
| Sodium Hydroxide | QS to adjust to pH 3.5-6.5 |
| Water for Injection | OS to 100 |

### Example 12

### Aqueous Injectable Formulations

| INGREDIENTS | % w/w |
|---|---|
| PQQ and/or derivative | 1.00 |
| AMP | 12.50 |
| UTP | 0.10 |
| Amino Acids | 3.0-7.0 |
| Polysorbate 80 | 0.40 |
| Sorbitol | 40.00 |
| Sodium Chloride | 0.90 |
| Benzyl alcohol | 0.90 |
| Buffer Sat(s) | QS to adjust to pH 3.5-6.5 |
| Sodium Hydroxide | QS to adjust to pH 3.5-6.5 |
| Water for Injection | QS to 100 |

### Example 13

### Aqueous Injectable Formulations

| INGREDIENTS | % w/w |
|---|---|
| PQQ and/or derivative | 2.10 |
| Polysorbate 80 | 0.40 |
| AMP | 12.50 |
| UTP | 0.10 |
| Amino Acids | 3.0-7.0 |
| Sodium Citrate | 0.50 |
| Sodium Chloride | 0.90 |
| Benzyl alcohol | 0.90 |
| Buffer Salt(s) | QS to adjust to pH 3.5-6.5 |
| Sodium Hydroxide | QS to adjust to pH 3.5-6.5 |
| Water for Injection | QS to 100 |

### Example 14

### Emulsion Injectable Formulations

| INGREDIENTS | % w/w |
|---|---|
| PQQ and/or derivative | 1.00 |
| AMP | 12.5 |
| UTP | 0.10 |
| Amino Acids | 3.0-7.0 |
| Sesame Oil or Glycerides | 2.0-12.0 |
| Polysorbate 80 | 0.40 |
| Sodium Chloride | 0.90 |
| Benzyl alcohol | 0.90 |
| Buffer Salt(s) | QS to adjust to pH 3.5-6.5 |
| Sodium Hydroxide | QS to adjust to pH 3.5-6.5 |
| Water for Injection | QS to 100 |

### Example 15

### Aqueous Injectable Formulations

| INGREDIENTS | % w/w |
|---|---|
| PQQ and/or derivative | 1.00 |
| AMP | 12.50 |
| UTP | 0.10 |
| Amino Acids | 3.0-7.0 |
| Olive Oil or Glycerides | 1.0-15.0 |
| Lecithin | 0.50-5.0 |
| Sorbitol | 30.00 |
| Sodium Chloride | 0.90 |
| Benzyl alcohol | 0.90 |
| Buffer Sat(s) | QS to adjust to pH 3.5-6.5 |
| Sodium Hydroxide | QS to adjust to pH 3.5-6.5 |
| Water for Injection | QS to 100 |

### Example 16

### Aqueous Injectable Formulations

| INGREDIENTS | % w/w |
|---|---|
| PQQ and/or derivative | 1.00 |
| Peanut Oil or Glycerides | 1.0-15.0 |
| Polysorbate 80 | 0.2-10.0 |
| AMP | 12.50 |
| UTP | 0.10 |
| Amino Acids | 3.0-7.0 |
| Sodium Citrate | 0.50 |
| Sodium Chloride | 0.90 |
| Benzyl alcohol | 0.90 |
| Buffer Salt(s) | QS to adjust to pH 3.5-6.5 |
| Sodium Hydroxide | QS to adjust to pH 3.5-6.5 |
| Water for Injection | QS to 100 |

### Example 17

### Gel Formulations

| INGREDIENTS | % w/w |
|---|---|
| PQQ and/or derivative | 1.00 |
| Peptides/Polypeptides | 3.00 |
| AMP | 12.50 |
| UTP | 0.10 |
| Propylene Glycol | 12.0 |
| Carbomer | 1.00 |
| Buffer Salt(s) | QS to pH 3.5-6.5 |
| Methylparaben | 0.20 |
| Propylparaben | 0.10 |
| Purified Water | QS to 100 |

### Example 18

### Gel Formulations

| INGREDIENTS | % w/w |
|---|---|
| PQQ and/or derivative | 1.00 |
| Peptides/Polypepetides | 3.00 |
| AMP | 12.50 |
| UTP | 0.10 |
| Glycerin | 5.00 |
| Hydroxyethylcellulose | 2.00 |
| Triethanolamine | QS to pH 3.5-6.5 |
| Methylparaben | 0.20 |
| Propylparaben | 0.10 |
| Purified Water | QS to 100 |

### Example 19

### Gel Formulations

| INGREDIENTS | % w/w |
|---|---|
| PQQ and/or derivative | 1.00 |
| Peptides/Polypepetides | 3.00 |
| AMP | 12.50 |
| UTP | 0.10 |
| Glycerin | 15.0 |
| Poloxamers 407/188 | 10.00 |
| Triethanolamine | QS to pH 3.5-6.5 |
| Methylparaben | 0.025 |
| Propylparaben | 0.015 |
| Purified Water | QS to 100 |

### Example 20

### Cream Formulations

| INGREDIENTS | % w/w |
|---|---|
| PQQ and/or derivative | 1.00 |
| White Petrolatum | 20.0 |
| Stearyl Alcohol | 20.0 |
| Propylene Glycol | 12.0 |
| Peptides/Polypeptides | 3.00 |
| AMP | 12.50 |
| UTP | 0.10 |
| Sodium lauryl sulfate | 1.00 |
| Methylparaben | 0.20 |
| Propylparaben | 0.10 |
| Buffer Salt(s) | QS to pH 3.5-6.5 |
| Purified Water | QS to 100 |

### Example 21

### Cream Formulations

| INGREDIENTS | % w/w |
|---|---|
| PQQ and/or derivative | 1.00 |
| Peptides/Polypeptides | 3.00 |
| AMP | 12.50 |
| UTP | 0.10 |
| Mineral Oil | 15.0 |
| Lanolin Alcohol | 10.0 |
| Cetyl Alcohol | 0.20 |
| Beeswax | 4.00 |
| Sorbitan Monoleate | 5.00 |
| Glycerin | 5.00 |
| Borax | 0.30 |
| Triethanolamine | 0.70 |
| Methylparaben | 0.20 |
| Propylparaben | 0.10 |
| Buffer Salt(s) | QS to pH 3.5-6.5 |
| Purified Water | QS to 100 |

### Example 22

### Cream Formulations

| INGREDIENTS | % w/w |
|---|---|
| PQQ and/or derivative | 1.00 |
| Peptides/Polypeptides | 3.00 |
| AMP | 12.50 |
| UTP | 0.10 |
| Glyceryl Monostearate | 10.0 |
| Lanolin | 2.00 |
| Glycerin | 10.0 |
| Stearyl Pyridinium Chloride | 1.50 |
| Methylparaben | 0.025 |
| Propylparaben | 0.015 |
| Buffer Salt(s) | QS to pH 3.5-6.5 |
| Purified Water | QS to 100 |

### Example 23

### Transdermal Systems

| INGREDIENTS | % w/w |
|---|---|
| PQQ and/or derivative | 5.00 |
| N-methylpyrrolidone | 15.0 |
| Peptides | 3.00 |
| AMP | 12.50 |
| UTP | 0.10 |
| Alcohol USP | 2.00 |
| Benzyl alcohol | 1.00 |
| Buffer Salt(s) | QS to pH 3.5-6.5 |
| Purified Water | QS to 100 |

### Example 24

### Transdermal Systems

| INGREDIENTS | % w/w |
|---|---|
| PQQ and/or derivative | 5.00 |
| Peptides | 3.00 |
| AMP | 12.50 |
| UTP | 0.10 |
| Ethoxydiglycol | 25.0 |
| Alcohol USP | 2.00 |
| PEG esters and monoglycerides | 15.0 |
| Benzyl alcohol | 1.00 |
| Buffer Salt(s) | QS to pH 3.5-6.5 |
| Purified Water | QS to 100 |

### Example 25

### Transdermal Systems

| INGREDIENTS | % w/w |
|---|---|
| PQQ and/or derivative | 5.00 |
| Peptides | 3.00 |
| AMP | 12.50 |
| UTP | 0.10 |
| Propylene Glycol | 25.0 |
| Alcohol USP | 4.00 |
| Polysorbate 80 | 10.0 |
| Benzyl alcohol | 1.00 |
| Buffer Salt(s) | QS to pH 3.5-6.5 |
| Purified Water | QS to 100 |

### Example 26

### Liquid Reservoir for Transdermal Patch

| INGREDIENTS | % w/w |
|---|---|
| PQQ and/or derivative | 5.00 |
| N-methylpyrrolidone | 10.0 |
| Peptides | 3.00 |
| AMP | 12.50 |
| UTP | 0.10 |
| Alcohol USP | 45.0 |
| Benzyl alcohol | 1.00 |
| Buffer Salt(s) | QS to pH 3.5-6.5 |
| Purified Water | QS to 100 |

### Example 27

### Liquid Reservoir for Transdermal Patch

| INGREDIENTS | % w/w |
|---|---|
| PQQ and/or derivative | 5.00 |
| Peptides | 3.00 |
| AMP | 12.50 |
| UTP | 0.10 |
| Ethoxydiglycol | 20.0 |
| Alcohol USP | 50.0 |
| Benzyl alcohol | 1.00 |
| Buffer Salt(s) | QS to pH 3.5-6.5 |
| Purified Water | QS to 100 |

### Example 28

### Liquid Reservoir for Transdermal Patch

| INGREDIENTS | % w/w |
|---|---|
| PQQ and/or derivative | 5.00 |
| Peptides | 3.00 |
| AMP | 12.50 |
| UTP | 0.10 |
| Propylene Glycol | 20.0 |
| Alcohol USP | 40.0 |
| Polysorbate 80 | 5.0 |
| Benzyl alcohol | 1.00 |
| Buffer Salt(s) | QS to pH 3.5-6.5 |
| Purified Water | QS to 100 |

A composition as provided herein may be administered chronically. As used herein, “chronically” has its normal meaning, which generally means repeated ingestion over a period of several days, several weeks or even several months. “Chronic” is not acute.

It should be understood that the examples and embodiments described herein are for illustrative purposes only and that various modifications or changes in light thereof will be suggested to persons skilled in the art and are to be included within the spirit and purview of this application and the scope of the appended claims.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

What is claimed is:

1. A composition useful for oral administration comprising:
one or more biologically-active forms of pyrroloquinoline quinone or PQQ derivatives;
one or more creatine species selected from the group consisting of creatine, creatine derivatives and creatyl peptides.

2. The composition of claim 1 further comprising coenzyme Q10 and melatonin.

3. The composition of claim 2 further comprising:
one or more amino acids;
one or more compounds selected from the group consisting of lipoic acid, ephedrine, caffeine, omega-3 fatty acids (fish oil), butein, piceatannol, fisetin, genistein, hydroxytyrosol, quercetin, isoliquiritigenin, casokinins, lactokinins and lactotripeptides;
one or more nucleic acid compounds selected from the group consisting of nucleotides, oligonucleotides, monophosphate nucleotides, diphosphate nucleotides, triphosphate nucleotides and cyclic derivatives of nucleotides; and
one or more triglycerides.

4. The composition according to claim 3, wherein said PQQ and/or its derivative is present in said composition in any amount between about 0.01% to about 10% by weight based on the total weight of said composition, including all percentages and ranges of percentages therebetween.

5. The composition according to claim 3 further comprising one or more nutritional adjuvant materials including flavoring agents, colorants, viscosity modifiers, preservatives, chelating agents, antioxidants, surface modifiers and other nutritional adjuvant materials.

6. The composition of claim 3 further comprising an anti-oxidant-containing food selected from the group consisting of cherries, bananas, grapes, rice and cereals, herbs, olive oil, wine, and beer.

7. The composition of claim 5 further comprising an anti-oxidant-containing food selected from the group consisting of cherries, bananas, grapes, rice and cereals, herbs, olive oil, wine, and beer.

8. The composition according to claim 3, wherein the creatine, or creatine derivatives are present in any amount between 0.001% and 20% by weight based on the total weight of said composition.

9. The composition according to claim 4 further comprising
one or more nutritional adjuvant materials including flavoring agents, colorants, viscosity modifiers, preservatives, chelating agents, antioxidants, surface modifiers and other nutritional adjuvant materials;
an anti-oxidant-containing food selected from the group consisting of cherries, bananas, grapes, rice and cereals, herbs, olive oil, wine, and beer;
wherein the creatine, or creatine derivatives are present in any amount between 0.001% and 20% by weight based on the total weight of said composition.

10. A method for activating the expression of SIRT genes, ACE genes and/or DAF 16 comprising the step of orally, intra-nasally or intra-ocularly administering a composition according to any one or more of claims 1-6 to a mammalian subject.
